(19) United States
(12) Patent Application Publication
(10) Pub. No.: US 2011/0209702 A1
(43) Pub. Date: Sep. 1, 2011

(54) Proportional Solenoid Valve For Low Molecular Weight Gas Mixtures

(75) Inventors: Steve Vuong, Vista, CA (US); David Winter, Encinitas, CA (US)

(73) Assignee: Nellcor Puritan Bennett LLC, Boulder, CO (US)

(21) Appl. No.: 12/713,439

(22) Filed: Feb. 26, 2010

(51) Int. Cl. A61M 16/20; A61M 16/12

(52) U.S. Cl. 128/203.12; 128/205.24

(57) ABSTRACT

This disclosure describes systems and methods for ventilating a patient with a gas mixture containing a low molecular weight gas, such as helium. The disclosure describes a novel proportional solenoid valve for controlling a low molecular weight gas mixture in a medical ventilator with reduced leakage.

### INTRODUCTION

Breathing devices such as medical ventilators and anesthetic apparatuses normally include an inspiratory side for supplying breathing gas toward a subject and an expiratory side for removing breathing gas from the subject. In the inspiratory side, an inspiration gas regulation device is situated to control flow of gas and/or pressure in the inspiratory side. The inspiratory side can also change and/or adjust the gas mixture concentrations sent to a patient during ventilation. The breathing device can receive pressurized gas from a compressor or centralized pressurized air source, such as wall outlet in a hospital. Often times, different gases or gas mixtures have separate sources or lines. Inspiration gas regulation devices can also be utilized to control the concentrations of the different gas sources received by the breathing device. A gas manifold can be utilized to combine the different regulated gases.

The inspiration gas regulation devices can be valves. Valves can be controlled pneumatically, mechanically or electromechanically. Electromechanical actuators such as solenoids or voice coil motors have been used.

However, typically utilized solenoid valves have a propensity leak when low density gases such as helium are utilized. This leakage makes it difficult to control the gas mixture delivered to the patient and is wasteful of the expensive, low density gas.

### SUMMARY

This disclosure describes systems and methods for ventilating a patient with a gas mixture containing a low molecular weight gas, such as helium. The disclosure describes a novel proportional solenoid valve for controlling a low molecular weight gas mixture in a medical ventilator with reduced leakage.

This disclosure also describes a medical ventilator system including: a processor; a source of heliox; and a proportional solenoid valve controlled by the processor and adapted to control the flow of the heliox from the heliox source. The proportional solenoid valve further includes: a seat; a poppet; and an elastomeric material adhering to at least one of the seat and the poppet to form an elastomeric seal when the proportional solenoid valve is closed.

Yet, another aspect of the disclosure describes a pneumatic system. The pneumatic system includes: a processor; a ventilation system including a patient circuit controlled by the processor; a pressure generating system controlled by the processor, the pressure generating system is adapted to generate a flow of breathing gas in the patient circuit; a source of heliox; and a proportional solenoid valve controlled by the processor and adapted to control the amount of the heliox delivered into the patient circuit. The proportional solenoid valve further includes: a seat; a poppet; and an elastomeric material adhering to at least one of the seat and the poppet to form an elastomeric seal when the proportional solenoid valve is closed.

These and various other features as well as advantages will be apparent from a reading of the following detailed description and a review of the associated drawings. Additional features are set forth in the description that follows and, in part, will be apparent from the description, or may be learned by practice of the described embodiments. The benefits and features will be realized and attained by the structure particularly pointed out in the written description and claims hereof as well as the appended drawings.

It is to be understood that both the foregoing general description and the following detailed description are exemplary and explanatory and are intended to provide further explanation of the claimed invention.

### BRIEF DESCRIPTION OF THE DRAWINGS

The following drawing figures, which form a part of this application, are illustrative of embodiments systems and methods described below and are not meant to limit the scope of the invention in any manner, which scope shall be based on the claims appended hereto.

FIG. 1 illustrates an embodiment of a ventilator connected to a human patient.

FIG. 2 illustrates an embodiment of a proportional solenoid valve for a ventilator.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. The reader will understand that the technology described in the context of a medical ventilator for human patients could be adapted for use with other systems such as ventilators for non-human patients and general gas transport systems in which periodic gas mixture changes may be required. As utilized herein a “gas mixture” includes at least one of a pure gas and a mixture of pure gases.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gas having a desired concentration of oxygen and other gases is supplied to the patient at desired pressures and rates. Ventilators capable of operating independently of external sources of pressurized air are also available.

While operating a ventilator, it can be desirable to add helium, heliox, or other gas mixtures with gas densities less than the density of air and/or oxygen to the breathing gas delivered to a patient. The gas density of helium is approximately 1/7^{th} of the density of air. Such gases are typically referred to as “low density” or “low molecular weight” gas mixtures. Low molecular weight gas mixtures are often expensive and used only under special circumstances.

Low molecular weight gas mixtures have the propensity to leak past most sealing interfaces that would otherwise be sufficiently effective for normal density gas mixtures. With air or oxygen gas, a metal-on-metal seat/poppet arrangement in a proportional solenoid valve is desirable for its clean, repeatable lift-off characteristics while maintaining reasonable leakage performance. For operation with low density gas mixtures, such as helium or heliox (a helium and oxygen gas mixture), however, a different sealing configuration is necessary due to the leakage allowed by a metal-on-metal seat/poppet arrangement.

Accordingly, a proportional solenoid valve for use with a low molecular weight gas mixture, such as helium or heliox is desirable. In one embodiment, a proportional solenoid valve for use with a low molecular weight gas mixture includes a poppet design with a thin but durable elastomeric material adhering on top of a metal substrate. The metal seat remains unchanged compared the conventional metal-on-metal seat/poppet arrangement. In an alternative embodiment, a proportional solenoid valve for use with low molecular weight gas mixture includes a seat design with a thin but durable elastomeric material adhering on top of a metal substrate. The metal poppet remains unchanged compared the conventional metal-on-metal seat/poppet arrangement. In another embodiment, a proportional solenoid valve for use with low molecular weight gas mixture includes a poppet and seat design both with a thin but durable elastomeric material adhering on top of a metal substrate.

With a soft material, a portion of the force budget for the valve is diverted from generating the opening for gas flow to sealing and compressing the elastomeric seal. A balance must be achieved in defining the thickness of the elastomeric material, the softness or durometer of the elastomeric material or sealing material, and the reduction in the effective stroke of the valve caused by the addition of the elastomeric material.

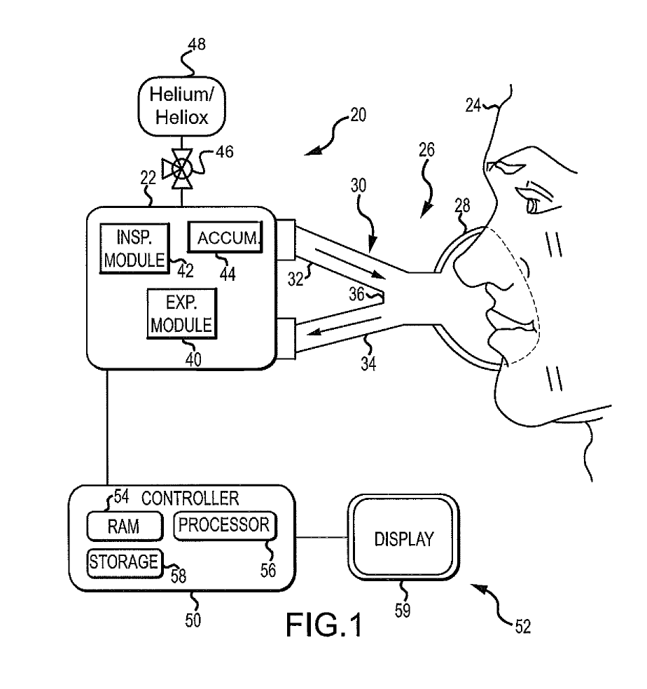

FIG. 1 illustrates an embodiment of a ventilator 20 connected to a human patient 24. Ventilator 20 includes a pneumatic system 22 (also referred to as a pressure generating system 22) for circulating breathing gases to and from patient 24 via the ventilation tubing system 26, which couples the patient 24 to the pneumatic system 22 via physical patient interface 28 and ventilator circuit 30. Ventilator circuit 30 could be a two-limb or one-limb circuit 30 for carrying gas mixture to and from the patient 24. In a two-limb embodiment as shown, a wye fitting 36 may be provided as shown to couple the patient interface 28 to the inspiratory limb 32 and the expiratory limb 34 of the circuit 30.

The present systems and methods have proved particularly advantageous in invasive settings, such as with endotracheal tubes. However, the present description contemplates that the patient interface 28 may be invasive or non-invasive, and of any configuration suitable for communicating a flow of breathing gas from the patient circuit 30 to an airway of the patient 24. Examples of suitable patient interface 28 devices include a nasal mask, nasal/oral mask (which is shown in FIG. 1), nasal prong, full-face mask, tracheal tube, endotracheal tube, nasal pillow, etc.

Pneumatic system 22 may be configured in a variety of ways. In the present example, system 22 includes an expiratory module 40 coupled with an expiratory limb 34 and an inspiratory module 42 coupled with an inspiratory limb 32. The inspiratory limb 32 receives a gas mixture from one or more gas sources 48 controlled by one or more gas regulators or gas regulation devices 46.

For instance, a helium/heliox gas source 48 and/or another source or sources of pressurized gas mixture (e.g., pressured air and/or oxygen) is controlled through the use of one or more gas regulators or gas regulation devices 46. In the embodiment shown, the gas regulator 46 includes a proportional solenoid valve for low density gases. As shown in FIG. 1, the gas regulator 46 is located within the ventilator 20. In one embodiment, the gas regulator 46 is located within the pneumatic system 22. In an alternative embodiment, the gas regulator 46 and/or proportional solenoid valve is a separate component independent of the ventilator 20.

In the illustrated embodiment, the gas regulator 46 and/or proportional solenoid valve is controlled by the ventilator 20. In one embodiment, the gas regulator 46 and/or proportional solenoid valve is controlled by the pneumatic system 22. In a further embodiment, the gas regulator 46 and/or proportional solenoid valve is controlled by the controller 50. In an alternative embodiment, the gas regulator 46 and/or proportional solenoid valve is controlled by a processor separate from and independent of the medical ventilator.

In the embodiment shown, the proportional solenoid valve has an elastomeric seal specific for low density gases. The elastomeric material may be any suitable material for substantially preventing a low molecular weight gas mixture from leaking through the proportional solenoid valve when closed. Accordingly, the processor for controlling the proportional solenoid valve for low density gases includes the information necessary to control the proportional solenoid valve for low density gases differently from the other valves to get accurate gas blends in the accumulator. In one embodiment, the proportional solenoid valve for low density gases includes lookup tables, formulae, logic, and etc. to control the proportional solenoid valve for low density gases differently from the other valves to get accurate gas blends in the accumulator.

Further, the gas concentrations can be mixed and/or stored in a chamber of a gas accumulator 44 at a high pressure to improve the control of delivery of respiratory gas to the ventilator circuit 30. The inspiratory module 42 is coupled to the helium/heliox gas source 48 and/or another gas mixture source, the gas regulator 46, and accumulator 44 to control the gas mixture of pressurized breathing gas for ventilatory support via inspiratory limb 32.

The pneumatic system 22 may include a variety of other components, including other sources for pressurized air and/or oxygen, mixing modules, valves, sensors, tubing, filters, etc. Controller 50 is operatively coupled with pneumatic system 22, signal measurement and acquisition systems, and an operator interface 52 may be provided to enable an operator to interact with the ventilator 20 (e.g., change ventilator settings, select operational modes, view monitored parameters, etc.). Controller 50 may include memory 54, one or more processors 56, storage 58, and/or other components of the type commonly found in command and control computing devices.

The memory 54 is computer-readable storage media that stores software that is executed by the processor 56 and which controls the operation of the ventilator 20. In an embodiment, the memory 54 comprises one or more solid-state storage devices such as flash memory chips. In an alternative embodiment, the memory 54 may be mass storage connected to the processor 56 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 56. Computer-readable storage media includes volatile and non-volatile, removable and non-removable media implemented in any method or technology for storage of information such as computer-readable instructions, data structures, program modules or other data. Computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the processor 56.

The controller 50 issues commands to pneumatic system 22 in order to control the breathing assistance provided to the patient 24 by the ventilator 20. The specific commands may be based on inputs received from patient 24, pneumatic system 22 and sensors, operator interface 52 and/or other components of the ventilator 20. In the depicted example, operator interface 52 includes a display 59 that is touch-sensitive, enabling the display 59 to serve both as an input user interface and an output device. The display 59 can display any type of ventilation information, such as sensor readings, parameters, commands, alarms, warnings, and smart prompts (i.e., ventilator determined operator suggestions).

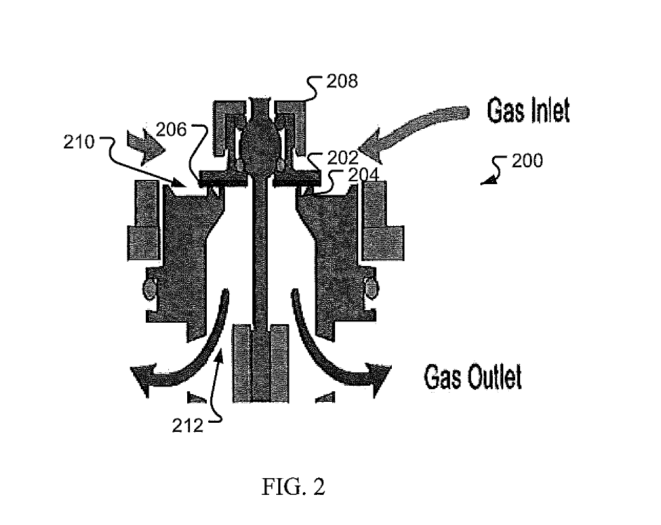

FIG. 2, illustrates an embodiment of a proportional solenoid valve 200 for low molecular weight gas mixture, such as in a ventilator 20 described above. The proportional solenoid valve 200 has an inlet 210 and an outlet 212 for breathing gas.

A valve seat 204 and a poppet 202 are arranged in the valve 200 to interact with each other for control of a valve opening, i.e. distance between valve seat 204 and poppet 202. In the embodiment shown, an elastomeric material 206 is adhered to the poppet 202. In an alternative embodiment, the elastomeric material 206 is adhered to the seat 204 of the proportional solenoid valve 200. In another embodiment, the elastomeric material 206 is adhered to both the seat 204 and the poppet 202 of the proportional solenoid valve 200.

The elastomeric material 206 may be any suitable material for preventing a low molecular weight gas mixture from substantially leaking through the proportional solenoid valve 200 when closed. In one embodiment, the elastomeric material 206 is selected from the group of silicone, viton, buna-N (Nitrile), ethylene propylene, and neoprene. In another embodiment, the elastomeric material 206 is selected from the group of butyl rubber, fluorocarbon, and polyurethane.

An actuator 208 controls the force exercised on the valve stem to move the poppet 202 away from the valve seat 204 depending on the control signal from a controller 50 (FIG. 1). As the poppet 202 moves away from the seat 204 the inlet 210 is opened allowing the gas mixture to flow into the proportional solenoid valve 200 and out of the proportional solenoid valve 200 through the outlet 212. By altering the force from the actuator 208, the flow in the inspiration tube from the gas source to the patient circuit can be controlled.

The actuator 208 also controls the force exercised on the poppet 202 to move it towards the valve seat 204 depending on the control signal from a controller 50 (FIG. 1) for compressing the elastomeric material 206 to seal the gas inlet 210. Further, depending upon the embodiment, such as the adhering of the elastomeric material 206 to the seat 204, poppet 202, and/or both, the thickness and the softness or the durometer of the elastomeric material 206 is specifically chosen to reduce and/or prevent a gas mixture with a molecular weight of less than air and/or oxygen from leaking through the proportional solenoid valve 200. Further, the addition of the elastomeric material 206 causes a reduction in the effective stroke of the proportional solenoid valve 200. As used herein “the effective stroke” of the proportional solenoid valve 200 is the distance the poppet 202 can move when acted upon by the actuator 208. In order to produce a proportional solenoid valve 200 that substantially reduces any leaking of a low molecular weight gas mixture, such as helium or heliox, a balance must be achieved in defining the thickness of the elastomeric material 206, the softness or durometer of the elastomeric material 206, and the reduction in the effective stroke of the proportional solenoid valve 200. As used herein, “substantially reduces any leaking” of the proportional solenoid valve 200 is when the amount of gas mixture leaked through the gas inlet 210 is less than or equal to about 0.010 standard liters per minute as measured with air under normal operating conditions. Air is utilized as the reference gas because flow sensors with helium calibration were not readily available.

Unless otherwise indicated, all numbers expressing quantities, properties, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

What is claimed is:

1. A medical ventilator system, comprising:
a processor;
a source of heliox; and
a proportional solenoid valve controlled by the processor and adapted to control the flow of heliox from the heliox source, the proportional solenoid valve comprising
a seat,
a poppet, and
an elastomeric material adhering to at least one of the seat and the poppet to form an elastomeric seal when the proportional solenoid valve is closed.

2. The medical ventilator system of claim 1, further comprising a gas manifold within the ventilator system connected to a patient circuit via a flow path, the gas manifold receiving a gas mixture from at least the source of heliox via the proportional solenoid valve.

3. The medical ventilator system of claim 2, further comprising an accumulator connected to the patient circuit downstream from the manifold.

4. The medical ventilator system of claim 1, wherein the source of heliox is selected from the group of a bottle and a wall source.

5. The medical ventilator system of claim 1, further comprising a source of at least one different gas mixture; a gas regulation device controlled by the processor and adapted to control the flow of the at least one different gas mixture delivered into the patient circuit.

6. The medical ventilator system of claim 1, wherein a force budget for the proportional solenoid valve is at least utilized for sealing and compressing the elastomeric seal.

7. The medical ventilator system of claim 1, wherein the proportional solenoid valve leaks less heliox than a proportional solenoid valve that utilizes metal-on-metal seat and poppet.

8. The medical ventilator system of claim 1, wherein a thickness of the elastomeric material, the durometer of the elastomeric material, and the reduction in the effective stroke of the proportional solenoid valve due to the addition of the elastomeric material are balanced to prevent more than about 0.010 standard liters per minute of air from leaking through the proportional solenoid valve.

9. The medical ventilator system of claim 1, wherein the elastomeric material is selected from a group of silicone, viton, buna-N, ethylene propylene, and neoprene.

10. A pneumatic system comprising:
a processor;
a ventilation system including a patient circuit controlled by the processor;
a pressure generating system controlled by the processor, the pressure generating system is adapted to generate a flow of breathing gas in the patient circuit;
a source of heliox; and
a proportional solenoid valve controlled by the processor and adapted to control the amount of the heliox delivered into the patient circuit, the proportional solenoid valve comprising
a seat,
a poppet, and
an elastomeric material adhering to at least one of the seat and the poppet to form an elastomeric seal when the proportional solenoid valve is closed.

11. The pneumatic system of claim 10, further comprising a gas manifold connected to the patient circuit via a flow path, the gas manifold receiving a gas mixture from at least the source of heliox.

12. The pneumatic system of claim 11, further comprising an accumulator connected to the patient circuit downstream from the manifold.

13. The pneumatic system of claim 10, wherein the source of heliox is selected from the group of a bottle and a wall source.

14. The pneumatic system of claim 10, further comprising a source of at least one different gas mixture; a gas regulation device controlled by the processor and adapted to control the amount of the at least one different gas mixture delivered into the patient circuit.

15. The pneumatic system of claim 10, wherein a force budget for the proportional solenoid valve is at least utilized for sealing and compressing the elastomeric seal.

16. The pneumatic system of claim 10, wherein the proportional solenoid valve leaks less heliox than a proportional solenoid valve that utilizes metal-on-metal seat and poppet.

17. The pneumatic system of claim 10, wherein a thickness of the elastomeric material, the durometer of the elastomeric material, and the reduction in the effective stroke of the proportional solenoid valve due to the addition of the elastomeric material are balanced to prevent more than about 0.010 standard liters per minute of air from leaking through the proportional solenoid valve.

18. The pneumatic system of claim 10, wherein the elastomeric material is selected from a group of silicone, viton, buna-N, ethylene propylene, and neoprene.
